(19) United States
(12) Patent Application Publication
(10) Pub. No.: US 2010/0069315 A1
(43) Pub. Date: Mar. 18, 2010

(54) METHOD AND MEDICAMENT FOR TREATING OCULAR INFECTIONS

(76) Inventors: Jacques Luyckx, Ceyrat (FR); Frederic Pilotaz, Chamalieres (FR); Pablo Goldschmidt, Paris (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/555,509

(22) Filed: Sep. 8, 2009

Related U.S. Application Data
(63) Continuation of application No. 11/408,161, filed on Apr. 21, 2006.

(30) Foreign Application Priority Data
Apr. 22, 2005 (FR) 0504069

(51) Int. Cl. A61K 31/7052; A61P 27/02

(52) U.S. Cl. 514/29

(57) ABSTRACT

The invention relates to a method for treating ocular infections, by topical application twice a day in each eye to be treated, for less than four days, of a medicament essentially consisting of azithromycin in solution in a pharmaceutically acceptable vehicle of linear medium-chain fatty acid triglycerides, at the concentration of 1% to 2%.

### BACKGROUND OF THE INVENTION

This application is a continuation application of U.S. Ser. No. 11/408,161, filed on Apr. 21, 2006.

The present invention relates to a method for the treatment and/or prevention of ocular infections with a medicament based on azithromycin. It also relates to a medicament based on azithromycin which is in a form corresponding to a complete therapy for the treatment of bacterial conjunctivitis, in particular of trachoma or of purulent conjunctivitis.

Azithromycin, or N-methyl-11-aza-10-deoxo-10-dihydroerythromycin, is an antibiotic of the macrolid class, which is known for its antibacterial activity and in particular for its spectrum of activity which is particularly suitable for the treatment of infections of the conjunctiva. In fact, most of the pathogenic species responsible for this type of infections exhibit sensitivity to this antibiotic.

In addition to conventional forms of conjunctivitis, or purulent conjunctivitis, azithromycin is particularly advantageous for the treatment of chlamydia conjunctivitis, due to the fact that these intracellular and atypical bacteria are highly sensitive to azithromycin. The chlamydia conjunctivitis, the frequency of which is probably underestimated in western countries, is transmitted either by direct contact with contaminated genital secretions (as in newborns), or indirectly, for example in a poorly disinfected swimming pool. In other regions of the world, in particular in hot countries (Africa, Asia, Middle East), trachoma, an infectious disease of the eye caused by the bacterium Chlamydia trachomatis, is hyperendemically rife. Trachoma is one of the main infectious causes of blindness and the main cause of ocular morbidity. There are therefore high stakes involved in being able to effectively treat bacterial conjunctivitis, in particular for trachoma in regions with uncertain sanitary conditions.

Document WO 021083178, published by the present inventors, describes ophthalmic compositions for topical application based on azithromycin as active ingredient. The azithromycin molecule is present therein in solution in an oily vehicle. The vehicle to be used preferably consists essentially of linear medium-chain fatty acid triglycerides (abbreviated to MCTs). The concentration of azithromycin is preferably between 0.7% and 2% by weight.

### SUMMARY OF THE INVENTION

The invention relates to a method for treating ocular infections, consisting in topically applying twice a day in each eye to be treated, for less than four days, a medicament essentially consisting of azithromycin, at the concentration of 1% to 2%, in solution in a pharmaceutically acceptable liquid vehicle of linear medium-chain fatty acid triglycerides.

The invention also relates to a medicament for the treatment of bacterial conjunctivitis, in particular of trachoma, which is provided in a box of four or six single-dose bottles each containing the amount necessary to be able to deliver 34 to 100 μl of a solution at a concentration of between 1% and 2% by weight of azithromycin dihydrate, in an oily vehicle essentially consisting of linear medium-chain fatty acid triglycerides and devoid of preserving agent.

### DETAILED DESCRIPTION OF THE INVENTION

The present invention comes from the discovery of the fact that the specific composition based on azithromycin in the concentration range of 1 to 2% by weight (expressed in terms of azithromycin dihydrate), and in solution in such an oily vehicle in the liquid phase, makes it possible to effectively treat infectious pathologies affecting the ocular sphere with a small number of doses distributed over a short period of treatment, advantageously less than 4 days.

It is clear that this particularity is of great importance for effective implementation of the treatments in high-risk regions where living conditions promote contamination, or on populations in which education or hygiene practices leave a lot to be desired.

The low number of daily doses is also an advantage under the same conditions. Insofar as the invention results in reducing the treatment to twice-daily instillations, there is much less risk of it being neglected and forgotten, while, furthermore, a patient who is cured after two or three days, at a pinch four, will have little tendency to stop the treatment prematurely.

In accordance with the invention, the two applications per day, in each eye requiring the treatment, are preferably spaced out over the day, one being in particular carried out in the morning and the other in the evening, on observing a gap of at least 6 hours between two administrations, in order to obtain the best effectiveness. Each application delivers a therapeutically effective dose of active ingredient under these conditions.

The prior art to the present invention emerges mainly from the series of patents by the applicant, already mentioned while referring to WO 02/083178. Other references exist, which are also directed towards azithromycin as active principle in topical ophthalmic treatments, but combining azithromycin with very different vehicles. The patent document US 2003/206956, from the company Insite Vision Incorporated, or the patent document EP 0925789, from the company Pfizer Products Inc., are thus found. In neither one case nor the other is azithromycin dissolved, and even less so dissolved in an oily medium. It is in solid form in suspension in an aqueous medium or in an ointment.

Furthermore, the authors thereof pretend that it should be possible to do with a low number of treatment doses in total for any value of azithromycin concentration in very broad concentration ranges, of from 0.01% to 2% or 2.5%, but the examples that they dare to provide do not go beyond a concentration of 0.5%, expressed in terms of azithromycin dihydrate. Furthermore, there is no indication of any result of a trial that can support the effectiveness of the treatment under these conditions. It has now been possible to observe that the hopes expressed in these patents were vain and that, in practice, the compositions in question do not make it possible to suitably treat ocular infections without the treatment being continued for at least 5 days.

In fact, MCTs represent an essential constituent in the preparation of the medicament according to the invention, as well as the solubilization of the azithromycin in these fatty acid triglycerides, which are in the liquid state under all the temperature and pressure conditions to which the product may normally be exposed. Another important factor for the effectiveness of the medicament and the success of the treatment under the conditions targeted concerns the concentration of the active ingredient in the solution, which can be precisely chosen, at values that provide this effectiveness under satisfactory solution stability and storage conditions.

Thus, according to the invention, the azithromycin is advantageously present at a concentration that remains within the range of 1% to 2%. In embodiments of the invention which give satisfaction in the vast majority of cases of application, whether in humans or in animals, this concentration is chosen at between 1% and 1.5%, in particular between 1.3% and 1.5%, and preferably in the region of 1.5%, as expressed by weight of azithromycin dihydrate relative to the total weight of the composition. However, it has been noted that the results observed in veterinary medicine in animals such as rabbits are not always directly transposable to humans or to other animal races, such as the ovine races or the bovine races. In the latter cases, and in particular in the case of trachoma in humans, use will often preferably be made of a concentration of 1.5% to 2% by weight, in particular of the order of 1.5% to 1.7% (weight of azithromycin dihydrate).

The vehicle chosen for the solubilization of the azithromycin is an oily liquid, a fatty oil with a low degree of unsaturation. It is easy to apply to the eye and the blurred vision after application is very clearly minimized compared to ointments. It remains present in tears and the conjunctiva for a long period of time, partly because the oily film that forms at the surface of the eye cannot be readily eliminated by tears.

In preferred embodiments of the invention, the MCT-type vehicle consists of esters of saturated fatty acids containing 5 to 18 carbon atoms, in particular of triglycerides of saturated fatty acids in which the alcohol functions of the glycerol are entirely esterified with acids of saturated hydrocarbons having a linear medium chain, such as capric acid (octanoic acid, C₈) and caprylic acid (decanoic acid, C₁₀).

In practice, the vehicle used is a fatty oil extracted from the species Cocos nucifera, refined and/or hydrogenated, which contains at least 95% of capric acid and/or caprylic acid. This vehicle, which is chemically very well defined, offers in particular the advantage of not being irritating for the eyes. It conventionally contains 50% to 80% of caprylic acid and 20% to 50% of capric acid. In preferred embodiments of the invention, a vehicle containing from 50% to 65% of caprylic acid and from 30% to 45% of capric acid is used.

Furthermore, the solutions of azithromycin in such a vehicle exhibit good stability, both chemically and physically. In particular, the medicaments prepared according to the invention offer the advantage of being able to be stored at ambient temperature, including when said temperature is high, up to 30° C., for example, for several months, without any degradation of the active ingredient being observed. This result is obtained even in the absence of preserving agent in the solution. Such a characteristic is entirely advantageous, and all the more so in the case of the treatment of trachoma, which is rife in hot countries, since it facilitates the operations of storage and transport of the medicaments obtained based on these solutions. Unlike other antibiotic ophthalmic compositions, it is not necessary to store the medicaments according to the invention in a refrigerated environment. They can be stored perfectly safely at ambient temperature, even under conditions where the outside temperature is high.

The medicament manufactured according to the invention is in particular to be administered during a maximum period of three days. It is particularly advantageous to administer it for a period of 2 days or 3 days, by twice-daily topical application.

Such a short treatment period had never been proposed in the prior art for topical ophthalmic treatment, whether for azithromycin or for any other antibiotic known to date. Now, such a treatment period is particularly advantageous, for several reasons.

Firstly, it reduces the risks of allergy or irritation associated with repeated administrations over long treatment periods. Secondly, the treatment is easy to carry out, since only a small number of administrations are necessary in total. The risk of the treatment being stopped too early, which is quite common in the case of treatments over a long period, which are more restrictive to carry out, is thus reduced. A short and simple treatment is also more suitable for modern life, whether for active individuals or in the case of the treatment of children in the community.

Furthermore, non-adherence to the dosages, and in particular stopping the treatment too early, promotes the selection of bacterial strains resistant to the antibiotic. This is all the more palpable in the hospital environment. Since the objective of a topical ophthalmic treatment is to treat a medium microbiologically contaminated with a set of potentially pathogenic microbes, it is easy to understand the importance of avoiding the risk of selection of a resistant strain, hence the need to scrupulously adhere to the effective dosage. A treatment over a short period of time, such as that proposed by the present invention, promotes adherence to the prescribed dosage.

The treatment as proposed by the present invention also has the advantages of topical application compared to an oral treatment. In particular, there is no need to drink in order to implement it. This characteristic is at its most important in the context of the treatment of trachoma, in hot countries where it is common for the water to be impure, and where the mere fact of drinking in order to absorb a medicament can cause other diseases.

The effectiveness of the treatment system according to the present invention for the treatment of bacterial conjunctivitis, in particular of trachoma or purulent conjunctivitis, is, as a result, entirely advantageous, in particular with regard to the prior art. The combination of azithromycin and the MCT-type vehicle under the conditions prescribed for administration twice a day (in particular in the morning when getting up and in the evening when going to bed) for less than four days, in particular two to three days, shows good effectiveness with respect to ocular infections. The small number of administrations results in the use of the antibiotic being limited to that strictly necessary for recovery, which corresponds to the requirements of the health authorities. In particular, a treatment period of 3 days, equivalent to 6 instillations in the eye in total (for each affected eye), for the curative treatment of purulent conjunctivitis, will be chosen. This treatment period can advantageously be shortened to only 2 days, equivalent to 4 instillations, for the eradication of trachoma (Chlamydia trachomatis eye infection).

In order to achieve another objective of the invention, concerning the ease with which the product is used and the absence of side effects, the invention provides a single-dose bottle packaging of the azithromycin-based ophthalmic medicament. This is intended to mean that, according to the embodiments of the invention, the product to be administered into the eye is presented contained in leaktight bottles of small volume and that these bottles each contain an amount of medicament sufficient for each application to be prescribed, and therefore in particular sufficient to be suitable for an application in each of the two eyes of the individual.

Each bottle receives an amount of composition sufficient to ensure that a therapeutically effective dose of active ingredient will be delivered into each eye during the instillation. This dose being preferably chosen to be at least approximately 0.24 mg per eye, each bottle contains, for example, a volume of composition corresponding at least to two drops, therefore in particular a volume of between 34 and 100 μl, i.e. 30 to 96 mg of the liquid consisting of the solution of azithromycin in the MCTs. By way of example, a bottle can contain approximately 400 μl of composition, which ensures that a drop, of the order of 17 to 50 μl in volume, i.e. 15 to 48 mg, may be administered in each eye. The single-dose bottle is disposable after administration: it is a single-use bottle.

Such a presentation provides many advantages. First of all, it makes it possible to readily deliver a therapeutically effective dose at each administration. Furthermore, in the hospital environment, it avoids the risks of inter-patient and intra-patient contamination by eliminating the risks of contamination via the nozzle of a bottle. This makes it possible, in combination with the good stability of the composition at ambient temperature, to obtain optimal hygiene conditions of use. This is all the more advantageous in the case of use in hot countries.

The single-dose bottle presentation form also makes it possible to do away with the use of a preserving agent in the composition. Thus, in preferred embodiments of the invention, the medicament is devoid of preserving agent, which advantageously decreases the risks of ocular irritation for users. The absence of preserving agent is also of value in facilitating an advantageous packaging of the medicament in bottles that can be readily produced in plastic. Thus, according to preferred embodiments of the invention, the single-dose bottles are individually produced in the form of plastic ampoules forming an application nozzle that ends with a neck that can be manually broken by the user.

The packaging in single-dose form, in combination with the small number of doses necessary for the treatment of eye infections (preferably 4 or 6 according to the invention), advantageously decreases the risks of misuse of the product, since all the doses are used for the treatment. It is advantageous, from an economical point of view, given the limited number of doses.

Along the same lines, the medicament for the treatment of bacterial conjunctivitis, in particular trachoma or purulent conjunctivitis, is advantageously provided in a form corresponding to a complete system for curative treatment. The packaging thereof according to the invention is advantageously in the form of a box of 4 to 6 single-dose bottles each containing the amount required to be able to deliver 34 to 100 microlitres (i.e. 30 to 96 mg) of a solution at approximately 1.5% by weight of azithromycin dihydrate in an oily vehicle essentially consisting of linear medium-chain fatty acid triglycerides. Preferably, no preserving agent is added.

Each bottle is advantageously a single-use bottle, i.e. the excess content after each application is to be thrown away and the user will open a new bottle for each application, whether the treatment prescribed relate to both eyes or to only one eye. The set is provided for topical administration in the eye at the rate of twice a day for 2 or 3 days. A period of 2 days is sufficient for the treatment of trachoma.

The medicament provided in such a packaging is particularly advantageous in the case of distribution circuits starting from production factories that adhere to strict hygiene requirements so as to serve regions that are difficult to access or populations with relatively negligent hygiene habits.

The invention will now be further specified in terms of its preferred characteristics and its advantageous results, through the detailed description of specific embodiments which are the subject of the examples hereafter. Unless otherwise stipulated, all the dimensions given in figures or other indications will be expressed, firstly, in accordance with international normalization and, secondly, as amounts by mass.

### EXAMPLE 1

An ophthalmic composition based on azithromycin at 1.5% by weight is prepared, corresponding to the following formula:

| | | |
|---|---|---|
| azithromycin dihydrate: | 1.5 | g |
| MCT: | q.s. 100 | g |

This composition contains no preserving agent. It is intended to be packaged in single-dose bottles.

The oily vehicle used corresponds to the definition of the European Pharmacopoeia monograph for a refined fatty oil containing 95% of medium chain triglycerides (MCTs) in which the alcohol functions of the glycerol are entirely esterified with carboxylic acids of saturated hydrocarbons the chain of which is linear and of medium length, i.e. essentially capric acid and/or caprylic acid. In the preferred embodiment that is the subject of the present example, the quality of medium-chain triglycerides used contains from 50% to 65% of caprylic acid and from 30% to 45% of capric acid.

A mass of MCT corresponding to 98% of the normal final mass is heated to 70° C. in a water bath. The azithromycin powder is dissolved in the triglycerides with stirring. The mixture is maintained at 70° C. for a few minutes, and then the solution obtained is left to cool to ambient temperature. The solution is then adjusted in terms of weight with the MCT, and the mixture is perfected for a few minutes with stirring.

The azithromycin remains in solution after cooling, and the solution obtained is light and clear. It is subjected to a sterilization treatment which is carried out by filtration. The procedure is carried out at ambient temperature. The filtration is carried out in a sterile environment, through a filter with a 0.2 μm mesh made of a polyethersulphone membrane. A true solution is obtained. This solution has a viscosity of 30 mPa·s (30 cPo at 20° C.). The density thereof is 0.95. The refraction index thereof is equal to 1.45. This value is very similar to that of tears, which is equal to 1.33 in a normal individual. As a result, the risk of blurred vision during instillation of the composition in the eye is minimal.

Stability studies were carried out in the following way. The solution was stored in the dark under temperature and humidity conditions, respectively, of 25° C. and 60%, to 40° C. and less than 25%. Samples were taken from the solution at various time points and analyzed by high pressure liquid chromatography in order to observe the degradation of azithromycin over time. The results show that the solution obtained remains stable over time, for a period of greater than six months at 40° C. There is no need to store it in a refrigerator in order to ensure its stability.

In its packaging, the solution is presented as remaining stable at temperatures that can range up to 30° C., for at least 24 months.

### EXAMPLE 2

An ophthalmic composition based on azithromycin at 1% by weight is prepared, in the same manner as for Example 1, corresponding to the following formula:

| | | |
|---|---|---|
| azithromycin dihydrate: | 1 | g |
| MCT: | q.s. 100 | g |

This composition contains no preserving agent. It is intended to be packaged in single-dose bottles.

### EXAMPLE 3

An ophthalmic composition based on azithromycin at 1,7% by weight is prepared, in the same manner as for Example 1, corresponding to the following formula:

| | | |
|---|---|---|
| azithromycin dihydrate: | 1.7 | g |
| MCT: | q.s. 100 | g |

This composition contains no preserving agent. It is intended to be packaged in single-dose bottles.

### EXAMPLE 4

Three ocular pharmacokinetic studies were carried out in animals. One drop, i.e. a maximum of 50 μl or 48 mg, of the composition of Example 1 was instilled in a rabbit's eye either once, or twice 12 hours apart, or four times at regular intervals over a period of 3 days.

Samples were taken from lachrymal fluid, the conjunctiva and the cornea, 8h, 12h, 24h, 2 days, 3 days and 6 days after the last instillation. The amount of azithromycin in each sample was determined by high performance liquid chromatography combined to mass spectrometry.

These three studies made it possible to specify the following elements:
After a single instillation of the composition of Example 1, the azithromycin levels are clearly higher than the critical concentration S described for azithromycin as being necessary to obtain therapeutic effectiveness at the ocular level, this being the case for at least 12 hours in the lachrymal film, 8 hours in the conjunctiva and 24 hours in the cornea.
After two instillations carried out 12 hours apart, these levels are much higher than the concentration S for at least 24 hours, following the final instillation, in the lachrymal film, for 8 hours in the conjunctiva and for 24 hours in the cornea.
After four instillations spaced out over three days, the azithromycin levels are higher than the concentration S for a further two days after the final instillation, for two thirds of the eyes treated, as regards the lachrymal film, and virtually all the eyes treated, in the conjunctiva. In the cornea, the levels are still much higher than the concentration S 6 days after the final instillation.

In conclusion, azithromycin administered to a rabbit's eye in the form of the composition of the example exhibit good distribution at the ocular surface and a prolonged residence time, at significant levels, in the superficial structures of the eye which are the targets for the therapeutic activity envisaged: lachrymal film, conjunctiva, cornea, this being after a small number of instillations over a short period of time.

The cornea in particular exhibits substantial azithromycin levels for a long time after the final instillation. It probably plays the role of a reservoir, in which the azithromycin concentrates at each instillation, and which gradually releases the azithromycin into the lachrymal film over time. This role of the cornea contributes to explaining the effectiveness of the treatment carried out with a small number of instillations over a short period of time.

### EXAMPLE 5

Three studies of the ocular toxicology of the composition of Example 1 were carried out in animals. These studies were aimed at determining the effects of this composition on corneal sensitivity in rabbits, acute ocular tolerance in rats, and ocular tolerance after repeated administration in rats.

Corneal sensitivity in rabbits: it was not modified after single administration of the composition of Example 1.

Acute ocular tolerance: the three compositions of Examples 1, 2 and 3 were tested after single instillation in the eye, according to the Draize method. None of the treatments caused ocular irritation.

Ocular tolerance with repeated administrations in rats:

Several studies were carried out using the compositions of the examples by means of instillations two or three times a day for 28 days, in comparison with a placebo (physiological saline) in the vehicle of the composition of Example 1, instilled 3 times a day.

The results showed good systemic tolerance of the treatments. In ocular terms, the multiple instillations of the composition at 1.5%, 1% and 1,7% of azithromycin dihydrate induced ocular irritation from the second week of treatment only (redness, chemosis, secretions). The histopathological ocular examinations revealed no lesion.

The results of the ocular toxicology studies showed that the composition according to the invention:

a) does not induce any corneal anaesthesia,

b) is not irritant,

c) does not induce any side effect under the recommended treatment conditions, since the drawbacks noted appear only for an instillation three times a day and not before the second week of treatment.

### EXAMPLE 6

Five clinical studies in humans were carried out with the compositions of Examples 1, 2 and 3 in normal individuals. In total, 260 individuals participated in these trials.

Two trials mainly studied the ocular tolerance of the three compositions in comparison with the vehicle alone after administration of one drop in a single eye. Three trials studied the ocular tolerance, and also the lachrymal and conjunctival pharmacokinetics, after single bilateral instillation or unilateral instillation repeated twice a day for one or three days.

The five studies made it possible to establish that, in normal individuals, the instillation of the compositions at the three concentrations studied is well tolerated by the ocular surface.

The most commonly reported ocular symptom following instillation was a burning and stinging sensation, beginning within 30 minutes after instillation and ending after a few minutes or a few hours. In these studies, the ocular symptoms observed in the vehicle group were similar. This makes it even more advantageous to limit, in accordance with the invention, the number of instillations of the composition and the period of treatment.

In these studies, the three concentrations tested were well tolerated, and no clinically relevant difference was observed between the treatment groups in terms of tolerance after ophthalmological examination of the ocular surface. In particular, in the study with unilateral instillations repeated twice a day for three days, no additional local adverse effect was observed. In all cases, no systemic side effect that could be attributed to the treatment was recorded.

Moreover, a pharmacokinetic study on normal individuals was carried out on 91 normal individuals who received a single instillation of a dose of the composition at 1.5% of azithromycin dihydrate in each eye, in comparison with a composition at 0.5%.

The azithromycin was assayed in the tears sampled 10 min 30 min, 2h, 4h, 8h, 12h and 24h after instillation.

The eye lotion at 1.5% is found to be highly superior to that of 0.5% in terms of value and duration of the concentration compared with the minimum inhibitory concentration for sensitive microbes and for microbes of intermediate sensitivity. The time periods for which effective concentrations are maintained are longer for the eye lotion at 1.5% of azithromycin dihydrate, with durations of always greater than 24 hours. Furthermore, the azithromycin levels in the tears are much lower for the eye lotion at 0.5%. In the latter case, the instillation of a single dose is insufficient to ensure maintenance of a therapeutically effective concentration of azithromycin in the lachrymal film for a few hours.

In yet another study, 36 normal individuals were given a drop of composition at 1.5% of azithromycin dihydrate, corresponding to one dose, in a single eye, twice a day for three days. The azithromycin was assayed in the tears sampled 12 hours after instillation of the final drop. The results obtained showed that the lachrymal levels were still relatively high at this late time.

The number of treated eyes exhibiting a lachrymal azithromycin level of greater than 0.5 μg/ml (maximum value of the critical threshold for sensitive microbes) or greater than 4 μg/ml (maximum value of the critical threshold for microbes of intermediate sensitivity), 12 hours after instillation of the final drop of the composition of Example 1, was, respectively, 12 out of 12 and 8 out of 12. With the eye lotion at 1%, these same proportions were, respectively, 11 out of 12 and 3 out of 12, therefore slightly less favourable.

The results of these lachrymal pharmacokinetic studies show that the posology of 1 to 2 drops, twice a day (morning and evening) for 3 days, of the composition according to the invention allows effective treatment of bacterial ocular infections.

In a fifth study also carried out on 36 individuals (3 groups of 12), the lachrymal and conjunctival concentrations of azithromycin were determined at even later times, i.e. 7 days (in the tears and the conjunctiva) and 14 days (in the conjunctiva) after the beginning of a treatment with instillations of 2 drops of the composition at 1.5% of azithromycin dihydrate for just one day or for 3 consecutive days. By way of comparison, a group of individuals were given 1 gram of azithromycin orally in a single dose.

The results of these studies reveal, following instillation of the composition according to the invention at the posology of 2 times one drop a day for 3 days (i.e., in total, approximately 2 mg of azithromycin administered), persistent conjunctival levels which are still notable 5 days after the instillations have been stopped, which ensures the effectiveness of this administration system, while at the same time avoiding the drawback of side effects, in particular in terms of ocular toxicity, and while providing all the advantages of a treatment of short duration.

1. A method for preventing trachoma, consisting essentially of topically applying twice a day in each eye, for less than four days, a medicament consisting essentially of azithromycin, at the concentration of 1% to 2%, in solution in a pharmaceutically acceptable liquid vehicle of linear medium-chain fatty acid triglycerides.

2. A method according to claim 1, wherein the azithromycin is present in solution in the linear medium-chain fatty acid triglycerides at a concentration of between 1% and 1.5% by weight of azithromycin dihydrate.

3. A method according to claim 2, wherein the azithromycin is present in solution in the linear medium-chain fatty acid triglycerides at a concentration of between 1.3% and 1.5% by weight of azithromycin dihydrate.

4. A method according to claim 1, wherein the azithromycin is present in solution in the linear medium-chain fatty acid triglycerides at a concentration of between 1.5% and 2% by weight of azithromycin dihydrate.

5. A method according to claim 1, wherein said medicament is administered for a period of only two or three days.

6. A method according to claim 5, wherein said medicament is administered in four instillations of one dose per eye over a period of two days.

7. A method according to claim 1, wherein said medicament is packaged in a single-dose eye drop bottle.

8. A method according to a claim 1, wherein said medicament does not contain any preserving agent.

9. A method for preventing trachoma in humans, consisting essentially of topically applying twice a day in each eye, over a period of only two or only three days, a medicament in which azithromycin is present at a concentration of between 1% and 2% by weight of azithromycin dihydrate, in solution in a vehicle consisting of linear medium-chain fatty acid triglycerides.

10-11. (canceled)

12. A method according to claim 1, wherein said solution is a true solution or a solution containing particles of not more than 0.2 microns.

13-14. (canceled)

15. A method according to claim 1, wherein the medicament is provided in a box of four or six single-dose eye drop bottles each containing the amount necessary to be able to deliver 34 to 100 μl of a solution at a concentration of between 1% and 2% by weight of azithromycin dihydrate, in an oily vehicle consisting essentially of linear medium-chain fatty acid triglycerides and devoid of preserving agent.

16-20. (canceled)

21. A method according to claim 15, in which the single-dose bottles are individually produced in the form of plastic ampoules forming an application nozzle that ends with a manually breakable neck.

22-33. (canceled)

34. A method according to claim 9, wherein the period is only two days.

35. A method according to claim 9, wherein the period is only three days.

36. A method according to claim 9, wherein the azithromycin is present in solution in the linear medium-chain fatty acid triglycerides at a concentration of between 1% and 1.5% by weight of azithromycin dihydrate.

37. A method according to claim 9, wherein the azithromycin is present in solution in the linear medium-chain fatty acid triglycerides at a concentration of between 1.3% and 1.5% by weight of azithromycin dihydrate.

38. A method according to claim 9, wherein the azithromycin is present in solution in the linear medium-chain fatty acid triglycerides at a concentration of between 1.5% and 2% by weight of azithromycin dihydrate.
